(19) United States
(12) Patent Application Publication
(10) Pub. No.: US 2024/0100243 A1
(43) Pub. Date: Mar. 28, 2024

(54) INTRAVENOUS SET WITH PUMP INTERFACE FOR RAPID INFUSION VIA REUSABLE DRIVER

(71) Applicant: CareFusion 303, Inc., San Diego, CA (US)

(72) Inventor: Ryan Callahan, Long Beach, CA (US)

(21) Appl. No.: 17/952,746

(22) Filed: Sep. 26, 2022

(51) Int. Cl. A61M 5/14; A61M 5/142; A61M 5/168

(52) U.S. Cl. CPC A61M 5/1413; A61M 5/142; A61M 5/16877

(57) ABSTRACT

An IV infusion set includes at least one infusion component, a first IV tube coupled to the infusion component, a second IV tube and a disposable pump head that. The pump head includes an inlet port coupled to the first IV tube, an outlet port coupled to the second IV tube and a drive interface configured to be coupled to a first end of a drive shaft. The drive interface is configured to be rotated by rotation of the drive shaft having a second end coupled to a driver to increase a fluid flow rate through the IV infusion set. IV set assemblies and methods of use are also provided.

### TECHNICAL FIELD

The present disclosure generally relates to intravenous (IV) sets, in particular to pump interfaces and reusable drivers for rapid infusion via IV sets.

### BACKGROUND

Medical treatments often include the infusion of a medical fluid (e.g., blood, plasma, saline) to patients using an IV catheter that is connected though an arrangement of flexible tubing and fittings, commonly referred to as an “IV set,” to a source of fluid, for example, an IV blood bag. During operation, medical fluid may be required quickly at greatly increased flow rates as shorter times to blood transfusions have been associated with decreased death risk in trauma patients. Typical IV sets use a cylindrical hand pump that is squeezed by hand to increase fluid flow rate, resulting in muscle fatigue and/or inadequate increases in fluid flow rates.

For these reasons, it is desirable to provide an IV set with a pump interface to be driven via a reusable mechanized driver to minimize muscle fatigue and to rapidly increase the volume of fluid pushed through the IV set.

The description provided in the background section should not be assumed to be prior art merely because it is mentioned in or associated with the background section. The background section may include information that describes one or more aspects of the subject technology.

### SUMMARY

In one or more embodiments, an IV infusion set comprises: at least one infusion component; a first IV tube coupled to the infusion component; a second IV tube; and a pump head that is disposable. The pump head comprises: an inlet port coupled to the first IV tube; an outlet port coupled to the second IV tube; and a drive interface configured to be coupled to a first end of a drive shaft, wherein the drive interface is configured to be rotated by rotation of the drive shaft having a second end coupled to a driver to increase a fluid flow rate through the IV infusion set.

In one or more embodiments, an IV set assembly comprises: at least one infusion component; a first IV tube coupled to the infusion component; a second IV tube; a driver; a drive shaft; and a pump head that is disposable. The pump head comprises: an inlet port coupled to the first IV tube; an outlet port coupled to the second IV tube; and a drive interface configured to be coupled to a first end of the drive shaft, wherein the driver is configured to rotate a second end of the drive shaft when the first end of the drive shaft is coupled to drive interface to increase a fluid flow rate out of the outlet port and through the second IV tube.

In one or more embodiments, a method of using an IV set assembly comprises: coupling a first end of an IV set to a fluid container; coupling a second end of the IV set to a catheter inserted into a patient; flowing fluid from the fluid container to the patient at a first fluid flow rate; coupling a driver via a drive shaft to a drive interface of a pump head of the IV set; operating the driver to rotate the drive shaft and the drive interface of the pump head; and flowing fluid from the fluid container to the patient at a second fluid flow rate greater than the first fluid flow rate based on the rotation of the drive interface of the pump head.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIG. 2 depicts a top view of a typical assembled IV infusion set with a hand pump.

FIG. 3 depicts a perspective view of an IV set with pump head, according to aspects of the disclosure.

FIG. 4 depicts a perspective view of the pump head of FIG. 3, according to aspects of the disclosure.

FIG. 5 depicts a perspective view of a pump head of an IV set, according to aspects of the disclosure.

FIG. 6 depicts a perspective view of a pump head of an IV set, according to aspects of the disclosure.

FIG. 7 depicts a perspective view of a driver, according to aspects of the disclosure.

FIG. 8 depicts a perspective view of an IV set with the pump head of FIG. 5 and the driver of FIG. 7, according to aspects of the disclosure.

FIG. 9 depicts a perspective view of the driver of FIG. 7 and the pump head of FIG. 5 connecting an infusion pump to a patient, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

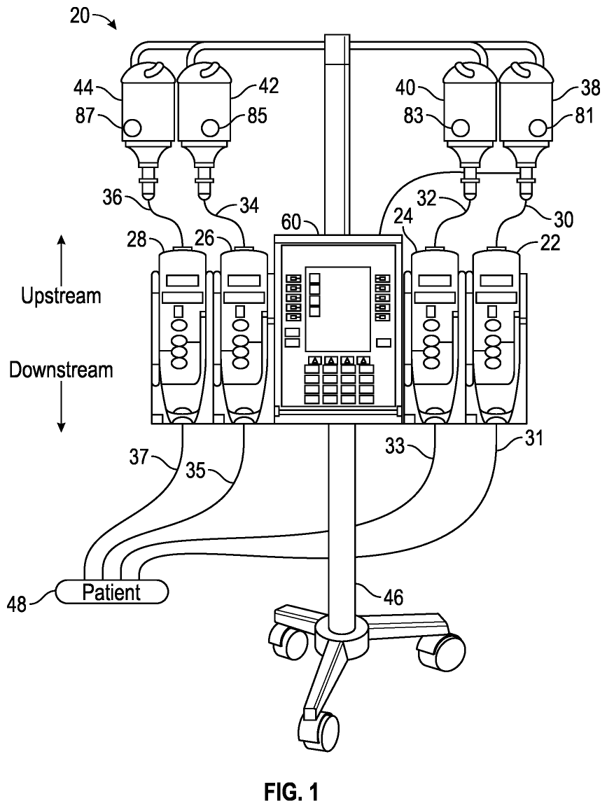

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

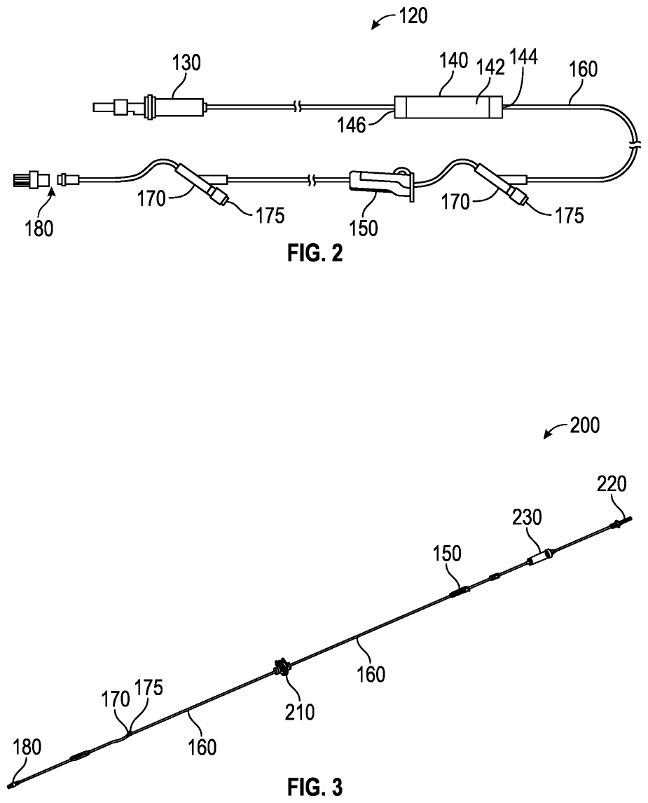

Typically, medical fluid administration sets have more parts than are shown in FIG. 1, such as those shown in FIG. 2. IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2, an IV set 120 may include a drip chamber 130, a hand pump 140 and a roller clamp 150 connected together by tubing 160. The IV set 120 may also include a Y-site 170 having a Y-shaped junction with a needleless connector 175, as well as a luer lock connector 180 at the end of the IV set 120. The luer lock connector 180 may be used for connection to a catheter inserted into a patient, for example. The IV set 120 may include additional infusion components and may be formed of any combination of components and the tubing 160.

In use, IV set 120 is connected to an IV fluid bag (e.g., blood bag) via the drip chamber 130 and the luer lock connector 180 is connected to a catheter that is placed into a vein of a patient. Thus, fluid flows from the fluid bag through the drip chamber 130 to the hand pump 140 and through the remainder of the IV set 120 and out of the luer lock connector 180. As the hand pump 140 is squeezed, the volume of fluid contained within a body 142 of the hand pump 140 is forced out an outlet port 144 and downstream through the IV set 120. When the hand pump 140 is released (e.g., stop squeezing), the body 142 of the hand pump 140 reinflates and draws in a new volume of fluid through an inlet port 146. This squeezing cycle can be repeated as often as necessary to move fluid from one or more fluid bags into the patient as quickly as possible.

The hand pump 140 is a typical flexible cylinder that is squeezed by a user continuously with one hand and released before activating or squeezing it again. However, the smooth cylindrical shape of the hand pump 140 provides a non-ergonomic shape to be gripped by a hand and no internal features to promote downstream fluid flow. Also, the squeezable cylinder shape geometry is often uncomfortable for prolonged use and may quickly lead to user hand fatigue. In addition, depending on the material the hand pump 140 is made of, it may take a long time to recoil and/or require extra squeeze strength to manipulate.

Often times there is a need to deliver intravenous fluids and/or blood rapidly. The subject technology facilitates rapid infusion of fluids/blood to a patient by including a pump interface (e.g., pump head) on the disposable IV set. The pump head may interface with a powered driver (e.g., a reusable driver) that turns a pump and delivers fluid. The pump head and driver may be configured to achieve almost any target flow rate (e.g., greater than 15 l/hr).

Typical infusion pumps are limited to 999 ml/hr, under a typical gravity (e.g., 36 in. h₂0) flow rate range from 1-5 l/hr depending on the catheter being used. A pressure cuff can increase the pressure to 300 mmhg and increase the flow rate to 4-8 l/hr. The subject technology can exceed all of these flow rates.

In aspects of the disclosure, a disposable pump head is integrated into the IV set. A pump interface may be engaged by a powered driver, which provides rotational input to the pump head. The pump head then delivers fluid downstream at a rapidly increased flow rate through the remaining IV set to the patient. This decreases or eliminates user hand fatigue from pumping fluid, such as with cylindrical hand pump 140, and rapidly speeds up the infusion rate in which new fluid is delivered to the patient. Also, unlike the cylindrical hand pump 140, the pump head and powered driver provide an increased fluid flow rate by rotating the driver interface faster instead of having to manually squeeze the hand pump more rapidly by hand, as well as providing a more continuous fluid flow as opposed to bursts of fluid flow.

In aspects of the disclosure, a pump head may be configured to be driven by any suitable drive shaft (e.g., hex shaped shaft similar to a powered screwdriver). For example, the pump head may be driven by an Intraosseous Driver, available from Becton Dickinson & Company. Here, the disposable pump head may be configured to interface with the Intraosseous Driver. In aspects of the disclosure, a disposable interface key may be included with the IV set, the disposable interface key configured to work with one or more specific powered drivers. In aspects of the disclosure, any suitable pump may be used (e.g., vane, peristaltic, gear, piston).

As shown in FIG. 3, an IV set 200 that includes a pump head 210 is provided according to aspects of the disclosure. For example, IV set 200 may include a spike 220, a drip chamber 230, a roller clamp 150 and the pump head 210 connected by tubing 160. The IV set 200 may also include a Y-site 170 having a Y-shaped junction with a needleless connector 175, as well as a luer lock connector 180 for connection to a catheter inserted into a patient, for example. The IV set 200 may include additional infusion components and may be formed of any combination of components and the tubing 160.

The pump head 210 may be formed of any suitable material (e.g., polymer, rubber, plastic) that is configured to be in contact with a medical fluid (e.g., saline, blood, plasma). The material of the pump head 210 may be flexible or inflexible and may be any single material or combination of materials selected or designed to provide a desired amount of flexibility/inflexibility and life-cycle usage.

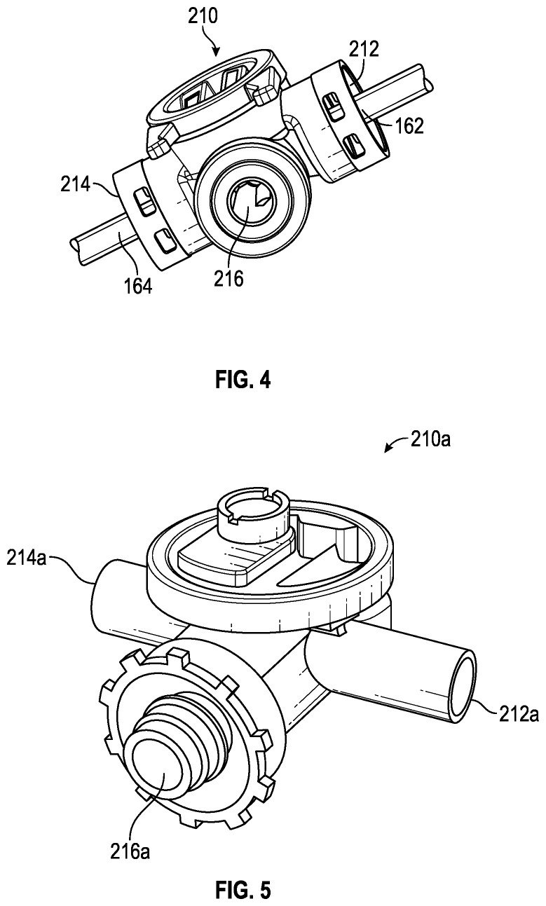

As shown in FIG. 4, in aspects of the disclosure, the pump head 210 may include an inlet port 212, an outlet port 214 and a drive interface 216. The inlet port 212 and outlet port 214 may be configured to receive a first tube end 162 and a second tube end 164, respectively. For example, the pump head 210 may be added to an existing IV set 200 by cutting the IV tube 160 in the desired location for the pump head 210, then inserting the first tube end 162 into the inlet port 212 and inserting the second tube end 164 into the outlet port 214. As another example, precut lengths of IV tube 160 may be connected to the pump head 210 during manufacturing/assembly of IV set 200. In aspects of the disclosure, the pump head 210 may be slidably moved onto IV tubing 160 or the IV tubing 160 may be fed into one of the inlet port 212/outlet port 214 and out the other of the inlet port 212/outlet port 214, thus leaving a portion of the IV tubing 160 extending through the pump head 210. The pump head 210 may be disposed of with the IV set 200 upon completion of use and/or end of life of the IV set 200 or any of the IV set 200 components. For example, pump head 210, 210a, 210b may be manufactured and/or used as a low cost disposable component.

FIG. 5 illustrates an embodiment of a pump head 210a. The pump head 210a may include an inlet port 212a, an outlet port 214a and a drive interface 216a. The inlet port 212a and outlet port 214a may be configured to receive a first tube end 162 and a second tube end 164, respectively. For example, the pump head 210a may be added to an existing IV set 200 by cutting the IV tube 160 in the desired location for the pump head 210a, then inserting the first tube end 162 into the inlet port 212a and inserting the second tube end 164 into the outlet port 214a. As another example, precut lengths of IV tube 160 may be connected to the pump head 210a during manufacturing/assembly of IV set 200. In aspects of the disclosure, the pump head 210a may be slidably moved onto IV tubing 160 or the IV tubing 160 may be fed into one of the inlet port 212a/outlet port 214a and out the other of the inlet port 212a/outlet port 214a, thus leaving a portion of the IV tubing 160 extending through the pump head 210a. The pump head 210a may be disposed of with the IV set 200 upon completion of use and/or end of life of the IV set 200 or any of the IV set 200 components.

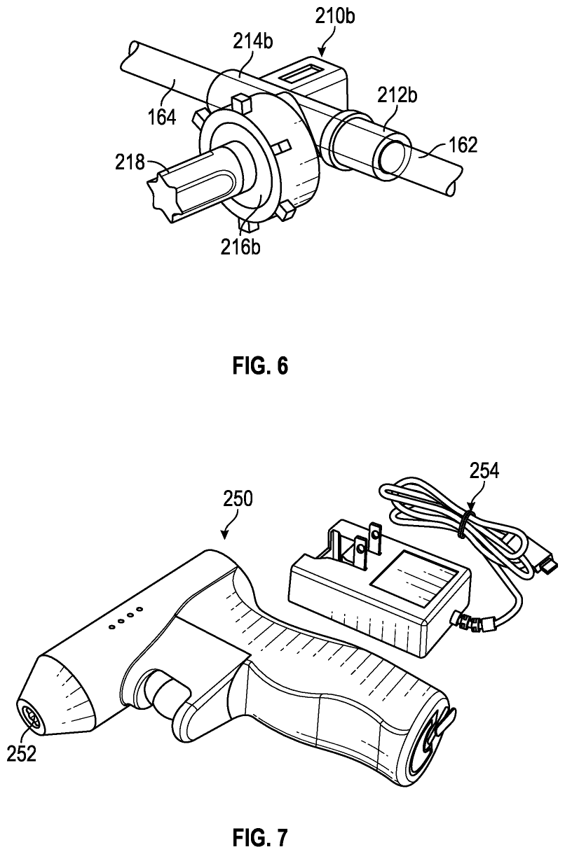

FIG. 6 illustrates an embodiment of a pump head 210b. The pump head 210b may include an inlet port 212b, an outlet port 214b and a drive interface 216b. The inlet port 212b and outlet port 214b may be configured to receive a first tube end 162 and a second tube end 164, respectively. For example, the pump head 210b may be added to an existing IV set 200 by cutting the IV tube 160 in the desired location for the pump head 210b, then inserting the first tube end 162 into the inlet port 212b and inserting the second tube end 164 into the outlet port 214b. As another example, precut lengths of IV tube 160 may be connected to the pump head 210b during manufacturing/assembly of IV set 200. In aspects of the disclosure, the pump head 210b may be slidably moved onto IV tubing 160 or the IV tubing 160 may be fed into one of the inlet port 212b/outlet port 214b and out the other of the inlet port 212b/outlet port 214b, thus leaving a portion of the IV tubing 160 extending through the pump head 210b. The pump head 210b may be disposed of with the IV set 200 upon completion of use and/or end of life of the IV set 200 or any of the IV set 200 components.

The drive interface 216, 216a, 216b may be sized and shaped to receive any suitable drive shaft (e.g., hex shaped shaft, star shaped shaft). For example, the drive interface 216, 216a, 216b may be configured to receive an end of a drive shaft 218, such as shown in the pump head 210b of FIG. 6. The other end of the drive shaft 218 may be received by a driver interface 252 of a driver 250 (see FIG. 7). In aspects of the disclosure, the drive shaft 218 may be coupled to the drive interface 216, 216a, 216b as an integral component of the pump head 210, 210a, 210b during manufacture/assembly of IV set 200. In aspects of the disclosure, the drive shaft 218 may be coupled to the drive interface 216, 216a, 216b before the IV set 200 is placed into service. In aspects of the disclosure, the drive shaft 218 may be coupled to the drive interface 216, 216a, 216b only as needed after the IV set 200 is placed into service. For example, one end of the drive shaft 218 may be coupled with the driver 250 and the other end of the drive shaft 218 may be inserted into the drive interface 216, 216a, 216b only when high flow rates via the pump head 210, 210a, 210b are needed and otherwise removed from the drive interface 216, 216a, 216b when regular flow rates through IV set 200 are desired. In aspects of the disclosure, the drive shaft 218 may be part of the pump head 210, 210a, 210b or of the driver 250, such that the driver 250 and the pump head 210, 210a, 210b interface directly. In aspects of the disclosure, the drive shaft 218 may be a separate disposable component that may be provided with the IV set 200.

As shown in FIG. 7, in aspects of the disclosure the driver 250 may be a rechargeable device configured to be charged by a charging assembly 254. In aspects of the disclosure, the driver 250 may have a power cord and be powered by plugging into a power source (e.g., wall socket). In aspects of the disclosure, the driver 250 may be configured to clip to any suitable structure (e.g., IV pole, hospital bed). In aspects of the disclosure, the driver 250 may be configured to operate at a fixed speed and/or at a variable speed.

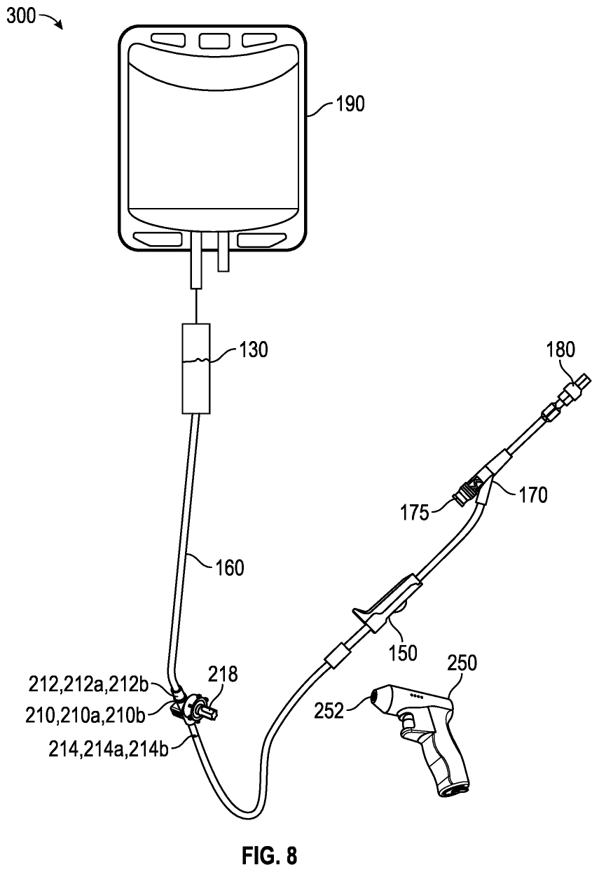

As shown in FIG. 8, the pump head 210, 210a, 210b may be part of an IV set 300. The IV set 300 may include a fluid bag 190, a drip chamber 130, the pump head 210, 210a, 210b, a roller clamp 150, a Y-junction 170 having a needleless port 175 and a luer connector 180, all coupled together by IV tubing 160.

In aspects of the disclosure, the pump head 210, 210a, 210b may be a passive in-line device when not activated (e.g., not being powered by the driver 250) in which fluid from the fluid bag 190 is received via the drip chamber 130 into the inlet port 212, 212a, 212b and the fluid passes through the pump head 210, 210a, 210b and out the outlet port 214, 214b, 214b at a flow rate controlled by any of gravity, the volume of fluid in the fluid bag 190 and/or the roller clamp 150. In use, when the drive shaft 218 is coupled to the drive interface 216, 216a, 216b and is rotated via the driver 250, the volume of fluid remaining upstream of the pump head 210, 210a, 210b from the passive flow phase may be forced through the pump head 210, 210a, 210b and out of the outlet port 214, 214b, 214b very rapidly over multiple revolutions of the drive shaft 218.

This rotation of the drive shaft 218 may cause a suction effect upstream in the IV set 300, thus causing more fluid to flow from the fluid bag 190/drip chamber 130 into the pump head 210, 210a, 210b quickly (e.g., more quickly than gravity allows). Thus, continuous rotation of the drive shaft 218 may cause all of the fluid to be rapidly pulled out of the fluid bag 190 and pushed into a desired receptacle (e.g., a patient's arm).

In aspects of the disclosure, the pump head 210, 210a, 210b may be a disposable component of the IV set 300. For example, the pump head 210, 210a, 210b may be an integral component of the IV set 300 where the pump head 210, 210a, 210b may be coupled in line via IV tubing 160 to the drip chamber 130 and the roller clamp 150. Accordingly, the pump head 210, 210a, 210b would be used for the life of the IV set 300 only (e.g., 24 hours, 72 hours, 7 days), whereafter the pump head 210, 210a, 210b would be disposed of along with the associated IV set 300. In aspects of the disclosure, the pump head 210, 210a, 210b may be coupled anywhere in line within the IV set 300, such as directly to the fluid bag 190 in place of a drip chamber 130 or close to the luer connector 180, for example.

In use, the IV set 300 with the disposable pump head 210, 210a, 210b is coupled to a fluid container (e.g., fluid bag 190) containing a medical fluid (e.g., blood). In an unactuated state, the pump head 210, 210a, 210b has a static drive interface 216, 216a, 216b and/or drive shaft 218, thus allowing the fluid to flow through the IV set 300 at a rate set by a flow controller (e.g., roller clamp 150). When quicker fluid flow is needed, the drive interface 216, 216a, 216b may be rotated by the drive shaft 218 coupled to the driver 250, thus driving the fluid through the IV set 300 more quickly.

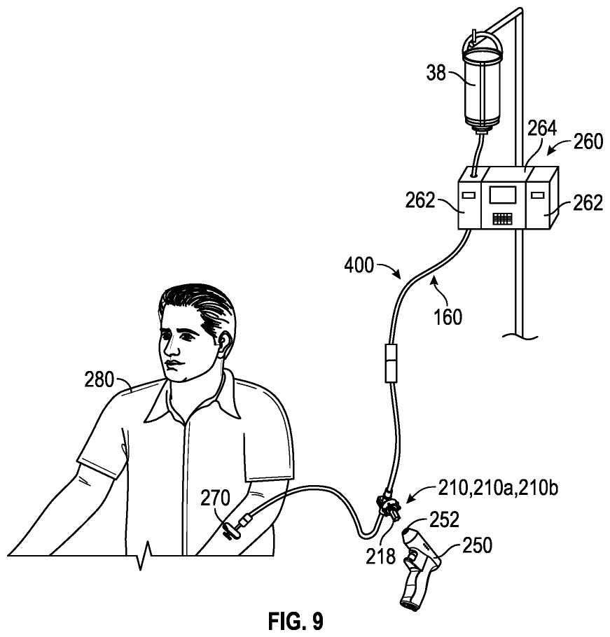

As shown in FIG. 9, an IV set 400 with pump head 210, 210a, 210b may be coupled to a fluid source 38, to an infusion pump 260 system having two infusion pumps 262 and a controller 264, and to a catheter 270 inserted into a patient 280. Here, if the maximum fluid flow rate from the infusion pump system 260 is not sufficient, the pump head 210, 210a, 210b may be activated (e.g., driven by driver 250) to force the fluid to flow more quickly. For example, the IV set 400 may be quickly uncoupled from the infusion pump system 260 before using the pump head 210, 210a, 210b and/or the infusion pump system 260 may be set to neutral to allow unimpeded fluid flow from the fluid source 38 before using the pump head 210, 210a, 210b.

In aspects of the disclosure, the pump head 210, 210a, 210b and/or the driver 250 may include any suitable fastener to couple the pump head 210, 210a, 210b and/or the driver 250 to an IV pole 46, a bed (e.g., hospital bed), a stretcher (e.g., an rollable ambulance stretcher), an operating table and the like. For example, the fastener may be a cradle, a hangar, a hook, Velcro®, adhesive, and/or any other suitable fastener. In aspects of the disclosure, the pump head 210, 210a, 210b may be configured to simply hang in line with an IV set (e.g., IV set 400) via IV tubing (e.g., IV tubing 160). Here, the pump head 210, 210a, 210b may be positioned on the IV set to maximize accessibility and/or to keep the pump head 210, 210a, 210b out of specific work areas (e.g., right above patient).

In aspects of the disclosure, drugs may be administered to a patient quickly via a pump head 210, 210a, 210b that forces the drugs via the IV line at the rate with which the drive shaft 218 is rotating.

In one or more embodiments, an IV infusion set comprises: at least one infusion component; a first IV tube coupled to the infusion component; a second IV tube; and a pump head that is disposable. The pump head comprises: an inlet port coupled to the first IV tube; an outlet port coupled to the second IV tube; and a drive interface configured to be coupled to a first end of a drive shaft, wherein the drive interface is configured to be rotated by rotation of the drive shaft having a second end coupled to a driver to increase a fluid flow rate through the IV infusion set.

In aspects of the disclosure, the drive shaft is provided as a removable component separated from the rest of the IV infusion set. In aspects of the disclosure, the drive shaft is a removable component pre-coupled to the pump head. In aspects of the disclosure, the drive shaft is an integral part of the pump head and the drive shaft is configured to be removably coupled to the driver. In aspects of the disclosure, the drive shaft is an integral part of the driver and the drive shaft is configured to be removably coupled to the pump head. In aspects of the disclosure, the drive interface is configured to receive a hex shaped drive shaft. In aspects of the disclosure, the drive interface is configured to receive a star shaped drive shaft. In aspects of the disclosure, the at least one infusion component comprises: a fluid connector configured to be coupled to a fluid container; and a luer lock connector configured to be coupled to a catheter inserted into a patient.

In one or more embodiments, an IV set assembly comprises: at least one infusion component; a first IV tube coupled to the infusion component; a second IV tube; a driver; a drive shaft; and a pump head that is disposable. The pump head comprises: an inlet port coupled to the first IV tube; an outlet port coupled to the second IV tube; and a drive interface configured to be coupled to a first end of the drive shaft, wherein the driver is configured to rotate a second end of the drive shaft when the first end of the drive shaft is coupled to drive interface to increase a fluid flow rate out of the outlet port and through the second IV tube.

In aspects of the disclosure, the drive shaft is removably coupled to each of the drive interface and to the driver. In aspects of the disclosure, the drive shaft is an integral part of the pump head and the drive shaft is removably coupled to the driver. In aspects of the disclosure, the drive shaft is an integral part of the driver and the drive shaft is removably coupled to the pump head. In aspects of the disclosure, the drive shaft is one of hex shaped and star shaped. In aspects of the disclosure, one of: the driver comprises a fastener configured to couple the driver to one of an IV pole, a bed, a stretcher and an operating table; and the pump head comprises a fastener configured to couple the pump head to one of an IV pole, a bed, a stretcher and an operating table. In aspects of the disclosure, the at least one infusion component comprises: a fluid connector configured to be coupled to a fluid container; and a luer lock connector configured to be coupled to a catheter inserted into a patient.

In one or more embodiments, a method of using an IV set assembly comprises: coupling a first end of an IV set to a fluid container; coupling a second end of the IV set to a catheter inserted into a patient; flowing fluid from the fluid container to the patient at a first fluid flow rate; coupling a driver via a drive shaft to a drive interface of a pump head of the IV set; operating the driver to rotate the drive shaft and the drive interface of the pump head; and flowing fluid from the fluid container to the patient at a second fluid flow rate greater than the first fluid flow rate based on the rotation of the drive interface of the pump head.

In aspects of the disclosure, the method comprises operating the driver to rotate the drive shaft and drive interface of the pump head at one of a fixed speed and a variable speed. In aspects of the disclosure, the method comprises one of: affixing a fastener of the driver to one of an IV pole, a bed, a stretcher and an operating table; and affixing a fastener of the pump head to one of an IV pole, a bed, a stretcher and an operating table. In aspects of the disclosure, the method comprises: coupling the IV set to an infusion pump; operating the infusion pump to flow the fluid from the fluid container to the patient at the first fluid flow rate; and before operating the driver to flow the fluid from the fluid container to the patient at the second fluid flow rate, one of: setting the infusion pump to neutral to provide unimpeded fluid flow through the infusion pump; and uncoupling the IV set from the infusion pump. In aspects of the disclosure, the first fluid flow rate is less than or equal to 999 ml/hr and the second fluid flow rate is greater than 15 l/hr.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) infusion set, comprising:
at least one infusion component;
a first IV tube coupled to the infusion component;
a second IV tube; and
a pump head that is disposable, the pump head comprising:
an inlet port coupled to the first IV tube;
an outlet port coupled to the second IV tube; and
a drive interface configured to be coupled to a first end of a drive shaft,
wherein the drive interface is configured to be rotated by rotation of the drive shaft having a second end coupled to a driver to increase a fluid flow rate through the IV infusion set.

2. The IV infusion set of claim 1, wherein the drive shaft is provided as a removable component separated from the rest of the IV infusion set.

3. The IV infusion set of claim 1, wherein the drive shaft is a removable component pre-coupled to the pump head.

4. The IV infusion set of claim 1, wherein the drive shaft is an integral part of the pump head and the drive shaft is configured to be removably coupled to the driver.

5. The IV infusion set of claim 1, wherein the drive shaft is an integral part of the driver and the drive shaft is configured to be removably coupled to the pump head.

6. The IV infusion set of claim 1, wherein the drive interface is configured to receive a hex shaped drive shaft.

7. The IV infusion set of claim 1, wherein the drive interface is configured to receive a star shaped drive shaft.

8. The IV infusion set of claim 1, wherein the at least one infusion component comprises:
a fluid connector configured to be coupled to a fluid container; and
a luer lock connector configured to be coupled to a catheter inserted into a patient.

9. An intravenous (IV) set assembly, comprising:
at least one infusion component;
a first IV tube coupled to the infusion component;
a second IV tube;
a driver;
a drive shaft; and
a pump head that is disposable, the pump head comprising:
an inlet port coupled to the first IV tube;
an outlet port coupled to the second IV tube; and
a drive interface configured to be coupled to a first end of the drive shaft,
wherein the driver is configured to rotate a second end of the drive shaft when the first end of the drive shaft is coupled to drive interface to increase a fluid flow rate out of the outlet port and through the second IV tube.

10. The IV set assembly of claim 9, wherein the drive shaft is removably coupled to each of the drive interface and to the driver.

11. The IV set assembly of claim 9, wherein the drive shaft is an integral part of the pump head and the drive shaft is removably coupled to the driver.

12. The IV set assembly of claim 9, wherein the drive shaft is an integral part of the driver and the drive shaft is removably coupled to the pump head.

13. The IV set assembly of claim 9, wherein the drive shaft is one of hex shaped and star shaped.

14. The IV set assembly of claim 9, wherein one of:
the driver comprises a fastener configured to couple the driver to one of an IV pole, a bed, a stretcher and an operating table; and
the pump head comprises a fastener configured to couple the pump head to one of an IV pole, a bed, a stretcher and an operating table.

15. The IV set assembly of claim 9, wherein the at least one infusion component comprises:
a fluid connector configured to be coupled to a fluid container; and
a luer lock connector configured to be coupled to a catheter inserted into a patient.

16. A method of using an intravenous (IV) set assembly, the method comprising:
coupling a first end of an IV set to a fluid container;
coupling a second end of the IV set to a catheter inserted into a patient;
flowing fluid from the fluid container to the patient at a first fluid flow rate;
coupling a driver via a drive shaft to a drive interface of a pump head of the IV set;
operating the driver to rotate the drive shaft and the drive interface of the pump head; and
flowing fluid from the fluid container to the patient at a second fluid flow rate greater than the first fluid flow rate based on the rotation of the drive interface of the pump head.

17. The method of claim 16, further comprising:
operating the driver to rotate the drive shaft and drive interface of the pump head at one of a fixed speed and a variable speed.

18. The method of claim 16, further comprising one of:
affixing a fastener of the driver to one of an IV pole, a bed, a stretcher and an operating table; and
affixing a fastener of the pump head to one of an IV pole, a bed, a stretcher and an operating table.

19. The method of claim 16, further comprising:
coupling the IV set to an infusion pump;
operating the infusion pump to flow the fluid from the fluid container to the patient at the first fluid flow rate; and
before operating the driver to flow the fluid from the fluid container to the patient at the second fluid flow rate, one of:
setting the infusion pump to neutral to provide unimpeded fluid flow through the infusion pump; and
uncoupling the IV set from the infusion pump.

20. The method of claim 19, wherein the first fluid flow rate is less than or equal to 999 ml/hr and the second fluid flow rate is greater than 15 l/hr.
